(19) United States
(12) Patent Application Publication
(10) Pub. No.: US 2021/0137858 A1
(43) Pub. Date: May 13, 2021

(54) COMBINATION FORMULATION CONTAINING COLCHICINE FOR TREATMENT OR ENHANCING THE THERAPY OF LIVER DISEASE

(71) Applicant: ACEBiomed, Inc., Jinju (KR)

(72) Inventor: Wang-Jo CHA, Gwacheon (KR)

(73) Assignee: ACEBiomed, Inc.

(21) Appl. No.: 16/914,276

(22) Filed: Jun. 27, 2020

(30) Foreign Application Priority Data
Nov. 13, 2019 (KR) 10-2019-0144930

(51) Int. Cl. A61K 31/165; A61K 31/235; A61P 1/16

(52) U.S. Cl. CPC A61K 31/165; A61P 1/16; A61K 31/235

(57) ABSTRACT

Exemplary embodiment of the present invention relate to a combination formulation for treatment or enhancing the therapy of liver disease, and more specifically, to a pharmaceutical composition for treatment or enhancing the therapy of liver disease which is prepared by combining drugs (biphenyldimethyl dicarboxylate, silymarin, ursodeoxycholic acid, etc.) which are known to have a conventional liver protective effect and colchicine to be administered in a combination formulation or in a combination form.

The composition according to embodiments of the present invention is a pharmaceutical composition for the treatment or enhancing the therapy of liver disease of a liver protective agent containing colchicine, and may be usefully used as a pharmaceutical composition for enhancing the therapy of liver fibrosis, liver cirrhosis, liver cancer or inflammatory liver disease including hepatitis by administering the colchicine in a single-dosage combination formulation or a separate-dosage combination form with the liver protective agent.

### CROSS-REFERENCE TO RELATED APPLICATIONS

This application claims priority from and the benefit of Korean Patent Application No. 10-2019-0144930, filed on Nov. 13, 2019, which is hereby incorporated by reference for all purposes as if fully set forth herein

### BACKGROUND

### Field

The present application relates to a combination formulation for treatment or enhancing the therapy of liver disease, wherein the combination formulation contains colchicine as a main active ingredient and may be prepared by combining at least one ingredient of DDB, silymarin, and UDCA according to the degree of liver disease. More specifically, the present application relates to a pharmaceutical composition for enhancing the therapy of liver disease which is prepared by specifically limiting the contents of drugs capable of exhibiting the best pharmacological effect when the drugs (biphenyldimethyl dicarboxylate, silymarin, ursodeoxycholic acid, etc.), which are known to have conventional liver protective effects, and colchicine is administered in a combination formulation or a combination form.

### Discussion of the Related Art

The liver is a biological organ that plays a pivotal role in the metabolism of substances out of the body and in the body, and continuously undergoes enzymatic reactions and energy metabolism. Currently, among the chronic diseases in Korea, hepatitis, liver cirrhosis, and liver cancer have the highest ratio with circulatory system diseases, and account for a large proportion of the causes of death from diseases. In particular, as the drinking population is larger, and the rate of liver damage caused by binge drinking is higher than in developed countries, the liver damage is of high interest. Continuous damage to liver tissues caused by viral infections or drinking has characteristics of disease that develop into liver cirrhosis or liver cancer. Considering the physiological characteristics and importance of the liver tissue, treatment and prevention of liver disease are very important, and there is a need to develop a pharmaceutical composition capable of reducing liver tissue damage and ultimately enhancing a therapy thereof.

In particular, liver fibrosis refers to a state in which a damaged liver tissue is transformed into a fibrous tissue such as collagen, rather than being repaired to normal hepatocytes, as part of a bioadaptation reaction accompanying chronic liver disease such as hepatitis. The liver fibrosis is a bio-adaptation reaction that occurs in the process of repairing tissue damage, but it is inevitable that liver functions deteriorate in a sense that the liver is replaced with fibrous tissues that cannot perform the intrinsic functions of a liver such as metabolism and bile secretion in vivo. The development of suitable therapeutic agents has been conducted as an important task in drug developments, in that the liver fibrosis phenomenon very often develops into liver cirrhosis, leading to death. Until now, however, since the mechanism of the liver fibrosis itself is not clearly known, no suitable therapeutic drug has been developed.

Meanwhile, colchicine has been widely used as a therapeutic agent for gout-related arthritis, and cases of successful treatment for acute gout and recurrent gout have been reported. In addition, the pharmacokinetic properties of colchicine have been also reported, and colchicine can be administered orally. For colchicine, it has been more than 10 years that cases for various indications have been reported, mechanism studies have been conducted, and colchicine has been reported to have effects on rheumatoid and non-rheumatic arthritis, prevention of amyloidosis in familial Mediterranean fever, prevention of fever, Behcet's disease, etc.

In this aspect, various attempts have been made for therapeutic agents for liver disease containing colchicine and a liver protective agent, but there are no studies that have confirmed that the therapy is enhanced even when a small amount of the liver protective agent is used due to a combination formulation containing colchicine or a co-administration of colchicine.

### SUMMARY

The present invention has been derived to solve the above problems, and the present inventors have confirmed that colchicine (which was a therapeutic agent for gouty arthritis) and biphenyldimethyl dicarboxylate (DDB), silymarin, and ursodeoxycholic acid (UDCA) (which are existing hepatocyte-protection and hepatitis anti-inflammatory agents) can be administered in combination to enhance the therapy of liver fibrosis or inflammatory liver disease, and completed the present invention based thereon.

Therefore, one object of the present invention is to provide a combination formulation for the treatment of liver disease containing colchicine and a liver protective agent.

Another object of the present invention is to provide a dose exhibiting the most suitable efficacy in administering colchicine and biphenyldimethyl dicarboxylate (DDB), silymarin, UDCA, and the like into the human body.

The objects of the present invention to be achieved are not limited to the aforementioned objects, and other objects, which are not mentioned above, will be apparently understood to those skilled in the art from the following description.

To achieve the objects of the present invention, exemplary embodiments of the present invention provide a combination formulation for the treatment of liver disease containing colchicine and a liver protective agent.

Exemplary embodiments of the present invention provide a pharmaceutical composition for enhancing the therapy of liver disease of a liver protective agent containing colchicine.

In one embodiment of the present invention, the pharmaceutical composition may be administered in a single-dosage combination formulation or a separate-dosage combination form with the liver protective agent.

In one embodiment of the present invention, the liver disease may be at least one selected from liver fibrosis, liver cirrhosis, liver cancer, and inflammatory liver disease.

In one embodiment of the present invention, the combination formulation for the treatment or enhancing the therapy of liver disease is composed of a combination of colchicine and at least one selected from biphenyldimethyl dicarboxylate (DDB), silymarin, and UDCA.

In one embodiment of the present invention, the co-administration may be in the form of administering the composition simultaneously, separately or sequentially with the liver protective agent.

In one embodiment of the present invention, the inflammatory liver disease may be selected from hepatitis, acute hepatitis, chronic hepatitis, alcoholic hepatitis, non-alcoholic hepatitis, subacute hepatitis, viral hepatitis, toxic liver disease, liver abscess, granulomatous hepatitis, autoimmune hepatitis, and lupus hepatitis.

In accordance with one embodiment of the present invention, a composition includes colchicine and at least one drug selected from biphenyldimethyl dicarboxylate (DDB), silymarin, and ursodeoxycholic acid (UDCA) and is administered in the combination formulation or the combination form to enhance the therapy of liver fibrosis or inflammatory liver disease.

In accordance with one embodiment of the present invention, colchicine and a liver protective agent are administered in the combination formulation or in combination to be usefully used or as the pharmaceutical composition for enhancing the therapy of liver disease such as liver fibrosis, liver cirrhosis, liver cancer or inflammatory liver disease including hepatitis even when the liver protective agent is administered in a low dose.

### BRIEF DESCRIPTION OF THE DRAWINGS

FIG. 1A illustrates results of albumin levels according to single administration of biphenyldimethyl dicarboxylate (DDB) and co-administration of colchicine and biphenyldimethyl dicarboxylate (DDB).

FIG. 1B illustrates results of albumin levels according to single administration of silymarin and co-administration of colchicine and silymarin.

FIG. 1C illustrates results of albumin levels according to co-administration of colchicine and UDCA.

FIG. 2A Illustrates results of measuring alkaline phosphate (ALP) according to single administration of biphenyldimethyl dicarboxylate (DDB) and co-administration of colchicine and biphenyldimethyl dicarboxylate (DDB).

FIG. 2B illustrates results of measuring alkaline phosphate (ALP) according to co-administration of colchicine and UDCA.

FIG. 3A Illustrates results of measuring ALT (GPT) according to single administration of biphenyldimethyl dicarboxylate (DDB) and co-administration of colchicine and biphenyldimethyl dicarboxylate (DDB).

FIG. 3B illustrates results of measuring ALT (GPT) according to single administration of silymarin and co-administration of colchicine and silymarin.

FIG. 3C illustrates results of measuring ALT (GPT) according to co-administration of colchicine and UDCA.

FIG. 4A Illustrates results of measuring AST (GOT) according to single administration of biphenyldimethyl dicarboxylate (DDB) and co-administration of colchicine and biphenyldimethyl dicarboxylate (DDB).

FIG. 4B illustrates results of measuring AST (GOT) according to single administration of silymarin and co-administration of colchicine and silymarin.

FIG. 4C illustrates results of measuring AST (GOT) according to co-administration of colchicine and UDCA.

FIG. 5A Illustrates results of total protein levels according to single administration of biphenyldimethyl dicarboxylate (DDB) and co-administration of colchicine and biphenyldimethyl dicarboxylate (DDB).

FIG. 5B illustrates results of total protein levels according to single administration of silymarin and co-administration of colchicine and silymarin.

FIG. 5C illustrates results of total protein levels according to co-administration of colchicine and UDCA.

FIG. 6A illustrates results of hydroxyproline assay according to single administration of biphenyldimethyl dicarboxylate (DDB) and co-administration of colchicine and biphenyldimethyl dicarboxylate (DDB).

FIG. 6B illustrates results of hydroxyproline assay according to single administration of silymarin and co-administration of colchicine and silymarin.

FIG. 6C illustrates results of hydroxyproline assay according to co-administration of colchicine and UDCA.

FIG. 7 illustrates results of liver histopathological examination (H&E staining) according to single administration of a liver protective agent and co-administration of colchicine and the liver protective agent.

### DETAILED DESCRIPTION OF EMBODIMENTS

The present inventors have confirmed that a combination formulation of colchicine and at least one ingredient selected from biphenyldimethyl dicarboxylate (DDB), silymarin, and ursodeoxycholic acid (UDCA) were administered to improve liver functions in patients with severe chronic active hepatitis with severe disease activity and early liver cirrhosis, have confirmed that colchicine combined or used in combination with a liver protective agent was administered to enhance the treatment, and completed the present invention based thereon

Hereinafter, exemplary embodiments of the present invention will be described in detail.

One exemplary embodiment of the present invention provides a combination formulation for the treatment of liver disease containing colchicine and a liver protective agent.

Another exemplary embodiment of the present invention provides a pharmaceutical composition for enhancing the therapy of liver disease of a liver protective agent containing colchicine.

In accordance with exemplary embodiments of the present invention, the pharmaceutical composition may be administered in a single-dosage combination formulation or a separate-dosage combination form with the liver protective agent.

In accordance with exemplary embodiments of the present invention, the liver disease may be at least one selected from liver fibrosis, liver cirrhosis, liver cancer, and inflammatory liver disease.

In accordance with exemplary embodiments of the present invention, the liver protective agent may be at least one selected from the group consisting of biphenyldimethyl dicarboxylate (DDB), silymarin, and ursodeoxycholic acid (UDCA).

In accordance with exemplary embodiments of the present invention, the administering of the composition in the separate-dosage combination form with the liver protective agent may include administering colchicine simultaneously, separately or sequentially with the liver protective agent.

The term “inflammatory liver disease” used in the present application is generally called hepatitis, and refers to an overall disease caused by inflammation of liver cells and liver tissue. The inflammatory liver disease according to embodiments of the present invention may include hepatitis, acute hepatitis, chronic hepatitis, alcoholic hepatitis, non-alcoholic hepatitis, subacute hepatitis, viral hepatitis, toxic liver disease, liver abscess, granulomatous hepatitis, autoimmune hepatitis, lupus hepatitis, etc., but is not limited thereto.

The term “treatment” used in the present application refers to any act of improving or beneficially altering liver fibrosis or inflammatory liver disease by administration of the pharmaceutical composition according to embodiments of the present invention.

In accordance with exemplary embodiments of the present invention, ‘biphenyldimethyl dicarboxylate (DDB)’ is an ingredient derived from shizandrae pulp as a hepatitis treating drug which is currently used clinically in East Asia including Korea. The biphenyldimethyl dicarboxylate (DDB) protects tissue damage by carbon tetrachloride, galactosamine, thioacetamide and prednisolone and increases antibody production. In addition, biphenyldimethyl dicarboxylate (DDB) is a drug that has therapeutic effects on actual hepatitis patients and is widely used clinically.

In accordance with exemplary embodiments of the present invention, ‘silymarin’ is a herbal extract obtained from the seeds of milk thistle, which is an asteraceae plant called white-patterned thistle. Silymarin is a drug that is frequently used for treating liver disease based on the principle of protecting liver cells by removing free oxygen.

In accordance with exemplary embodiments of the present invention, the ursodeoxycholic acid (UDCA) is a major ingredient of the gall bladder of a bear and has a strong detoxifying ability to activate detoxification and metabolism of the liver and prevent cholesterol from being accumulated in the liver and thus has been used as a therapeutic agent of liver disease.

The present inventors have confirmed through various experiments that the efficacy of treating liver disease is enhanced when colchicine and biphenyldimethyl dicarboxylate (DDB), silymarin, and UDCA are administered in combination.

In one embodiment of the present invention, through a blood biochemical test, as a result of confirming the therapy when colchicine, and biphenyldimethyl dicarboxylate (DDB), silymarin, or UDCA are administered in a combination formulation or in a combination form, as compared to single administration of biphenyldimethyl dicarboxylate (DDB), silymarin or UDCA, the recovery of albumin levels and total protein concentrations was confirmed, and a significant reduction effect was confirmed in ALP (alkaline phosphatase), ALT (GPT), AST (GOT), etc.

In addition, in one embodiment of the present invention, through a hydroxyproline assay, as a result of confirming fibrin levels when colchicine, and biphenyldimethyl dicarboxylate (DDB), silymarin, or UDCA are administered in a combination formulation or in a combination form, as compared to single administration of biphenyldimethyl dicarboxylate (DDB), silymarin or UDCA, the reduction of the fibrin levels was confirmed in the co-administration.

In addition, in one embodiment of the present invention, through a histopathological examination and H&E staining, as a result of confirming changes in liver functions and liver tissues when colchicine, and biphenyldimethyl dicarboxylate (DDB), silymarin, or UDCA are administered in a combination formulation or in a combination form, as compared to single administration of biphenyldimethyl dicarboxylate (DDB), silymarin or UDCA, further improvement of a pathology was confirmed in the co-administration.

In the combination formulation including colchicine as a main active ingredient and at least one drug selected from biphenyldimethyl dicarboxylate (DDB), silymarin, and ursodeoxycholic acid (UDCA) as the liver protective agent, the contents of the ingredients according to embodiments of the present invention are as follows.

When administering the combination formulation in accordance with embodiments of the present invention, one may use the content of colchicine of 10 to 120 μg/kg, the content of the biphenyldimethyl dicarboxylate (DDB) of 5 to 30 mg/kg, the content of the silymarin of 10 to 50 mg/kg, and the content of the ursodeoxycholic acid (UDCA) of 10 to 100 mg/kg.

When the content of the colchicine is less than 10 μg/kg, the efficacy of colchicine may be rarely exhibited, and when the content thereof is more than 120 μg/kg, there may be side effects of causing gastrointestinal disorders when administered.

When the content of the biphenyl dimethyl dicarboxylate (DDB) is less than 5 mg/kg, the efficacy may not be exhibited, and when the content thereof is more than 30 mg/kg, there may be side effects such as a skin rash, abdominal pain, and diarrhea.

When the content of silymarin is less than 10 mg/kg, the efficacy may not be exhibited, and when the content thereof is more than 50 mg/kg, side effects such as gastrointestinal disorders may occur.

When the content of ursodeoxycholic acid is less than 10 mg/kg, the efficacy may not be exhibited, and when the content thereof is more than 100 mg/kg, side effects such as diabetes may occur.

The term “combination formulation” used in the present application refers to a pharmaceutical composition provided according to embodiments of the present invention.

The composition according to embodiments of the present invention may further include suitable carriers, excipients and diluents, which are commonly used in the preparation of the pharmaceutical composition. In addition, according to general methods, the composition may be formulated and used in the form of oral formulations such as powders, granules, tablets, capsules, suspensions, emulsions, syrups, and aerosols, external preparations, suppositories and sterile injectable solutions, and may also be formulated and used in the form of a unit-dosage form suitable for oral administration.

The carrier, the excipient, and the diluent which may be included in the composition include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, mineral oil, etc. When the composition is formulated, the formulation may be prepared by using a diluent or an excipient, such as a filler, an extender, a binder, a wetting agent, a disintegrating agent, and a surfactant which are generally used.

A solid formulation for oral administration includes a tablet, a pill, a powder, a granule, a capsule, and the like, and the solid formulation may be prepared by mixing at least one excipient, for example, starch, calcium carbonate, sucrose, lactose, gelatin, and the like with the composition. Further, lubricants such as magnesium stearate and talc may be used in addition to simple excipients. A liquid formulation for oral administration may correspond to a suspension, an oral liquid, an emulsion, a syrup, and the like, and may include various excipients, for example, a wetting agent, a sweetener, an aromatic agent, a preserving agent, and the like in addition to water and liquid paraffin which are commonly used as simple diluents. A formulation for parenteral administration includes a sterile aqueous solution, a non-aqueous solution, a suspension, an emulsion, a lyophilizing agent, and a suppository. As the non-aqueous solution and the suspension, propylene glycol, polyethylene glycol, vegetable oil such as olive oil, injectable ester such as ethyl oleate, and the like may be used. As a base compound of the suppository, witepsol, macrogol, tween 61, cacao butter, laurin, glycerogelatin, and the like may be used.

In addition, an antioxidant may be further added to the composition for the treatment of liver disease and the like provided in the present invention. As the antioxidant, compounds in Vitamin B group such as thiamin (vitamin B1), riboflavin (vitamin B2), niacin (vitamin B3), pantothenic acid (vitamin B5), pyridoxine (vitamin B6), and cobalamin (vitamin B12), vitamin C, vitamin D, vitamin E, etc. may be used.

The pharmaceutical composition according to embodiments of the present invention may be administered in a pharmaceutically effective dose. In the present application, the “pharmaceutically effective dose” refers to a sufficient amount to treat disease at a reasonable benefit/risk ratio applicable to medical treatment. An effective dose level may be determined according to elements including the type and severity of disease of a patient, activity of a drug, sensitivity to a drug, a time of administration, a route of administration, an emission rate, duration of treatment, and simultaneously used drugs, and other elements well-known in the medical field. The pharmaceutical composition according to embodiments of the present invention may be administered as a separate therapeutic agent or in combination with other therapeutic agents, and may be administered sequentially or simultaneously with conventional therapeutic agents, and may be administered singly or multiply. It is important to administer an amount capable of obtaining a maximum effect with a minimal amount without side effects by considering all the elements, which may be easily determined by those skilled in the art.

The pharmaceutical composition according to embodiments of the present invention may be administered to a subject by various routes. All modes of administration may be expected, for example, the pharmaceutical composition may be administered by oral, rectal or intravenous, intramuscular, subcutaneous, intrathecal epidural or cerebrovascular injection. The pharmaceutical composition of the present invention may be determined according to a type of drug as an active ingredient in addition to many related factors, such as disease to be treated, the administration route, the age, gender, and weight of the patient, and the severity of the disease.

Hereinafter, Examples are presented in order to assist understanding of the present invention. However, the following Examples are just provided to more easily understand the present invention and the contents of the present invention are not limited by the following Examples.

### Example 1. Experiment Preparation and Experiment Method

1-1. Preparation of Experimental Animal

Male Wistar Rat [about 150 g, 4 to 5-week old]

1-2. Drugs and Administration Plan

An administration plan of carbon tetrachloride (CCl₄) and colchicine used in this experiment is as follows:

\textcircled{1} Carbon tetrachloride: Intraperitoneal administration of 0.4 g/kg·bw, administered 3 times per week for 10 weeks
\textcircled{2} Colchicine: Oral administration of 10, 50, 100 μg/kg·bw, once daily for 10 weeks

1-3. Classification of Experimental Groups

For this experiment, each group was classified as follows, and one group was used by 12 rats.

\textcircled{1} Control group (distilled water-administered group)

\textcircled{2} Vehicle-administered group (group administered with olive oil alone which is a CCl₄ solvent)

\textcircled{3} CCl₄-administered group [0.4 g/kg]

\textcircled{4} CCl₄+Colchicine [10 μg/kg]

\textcircled{5} CCl₄+Colchicine [50 μg/kg]

\textcircled{6} CCl₄+Colchicine [100 μg/kg]

1-4. Measurement Items

The following items were measured to confirm the enhancement of the efficacy for treating liver fibrosis. Fibrosis index items were measured at 10 weeks.

\textcircled{1} Fibrosis index item: hydroxyproline

### Example 2. Confirmation of Enhancement of Efficacy for Treating Liver Fibrosis According to Dose of Colchicine

2-1. Hydroxyproline Assay Quantitation

Hydroxyproline assay was performed for each group to confirm the levels of fibrin accumulation in the liver.

TABLE 1

| Group | Hydroxyproline [μg/g of liver] |
|---|---|
| Normal | 274.81 ± 17.49 |
| Vehicle | 247.86 ± 23.10 |
| CCl₄ [0.4 g/kg] | 336.36 ± 18.82 |
| CCl₄ + Colchicine [10 μg/kg] | 320.12 ± 13.32 |
| CCl₄ + Colchicine [50 μg/kg] | 313.33 ± 12.50 |
| CCl₄ + Colchicine [100 μg/kg] | 302.25 ± 10.25 |

As shown in Table 1 above, liver fibrosis by CCl₄ exhibited a significant reduction effect of fibrin in the colchicine-administered group.

### Example 3. Experiment Preparation and Experiment Method

3-1. Preparation of Experimental Animals

Male Wistar Rat [about 150 g, 4 to 5-week old]

3-2. Drugs and Administration Plan

An administration plan of carbon tetrachloride (CCl₄), silymarin, colchicine, and biphenyldimethyl dicarboxylate (DDB) used in this experiment is as follows:

\textcircled{1} Carbon tetrachloride: Intraperitoneal administration of 0.4 g/kg·bw, administered 3 times per week for 10 weeks

\textcircled{2} Silymarin: Oral administration of 10, 30, 50 mg/kg·bw, administered once a day for 10 weeks

\textcircled{3} Colchicine: Oral administration of 10 μg/kg·bw, administered once a day for 10 weeks

\textcircled{4} Biphenyldimethyl dicarboxylate (DDB): Oral administration of 10, 20, 25 mg/kg·bw, administered once a day for 10 weeks

\textcircled{5} UDCA: Oral administration of 30 mg/kg·bw, administered once a day for 10 weeks

3-3. Classification of Experimental Groups

For this experiment, each group was classified as follows, and one group was used by 12 rats.

\textcircled{1} Control group (distilled water-administered group)

\textcircled{2} Vehicle-administered group (group administered with olive oil alone which is a CCl₄ solvent)

\textcircled{3} CCl₄-administered group [0.4 g/kg]

\textcircled{4} CCl₄+DDB [25 mg/kg] administered group

\textcircled{5} CCl₄+Colchicine [10 μg/kg]+DDB [25 mg/kg] administered group

\textcircled{6} CCl₄+Colchicine [10 μg/kg]+DDB [20 mg/kg] administered group

\textcircled{7} CCl₄+Colchicine [10 μg/kg]+DDB [10 mg/kg] administered group

\textcircled{8} CCl₄+Silymarin [50 mg/kg] administered group

\textcircled{9} CCl₄+Colchicine [10 μg/kg]+Silymarin [50 mg/kg] administered group

\textcircled{10} CCl₄+Colchicine [10 μg/kg]+Silymarin [30 mg/kg] administered group

\textcircled{11} CCl₄+Colchicine [10 μg/kg]+Silymarin [10 mg/kg] administered group

\textcircled{12} CCl₄+Colchicine [10 μg/kg]+UDCA [30 mg/kg] administered group

3-4. Measurement Items

The following items were measured to confirm the enhancement of the efficacy for the treatment of liver disease (hepatitis, liver fibrosis, etc.). Serum items were measured at 4 weeks and 10 weeks, and histological items were measured at 10 weeks.

\textcircled{1} Serum enzyme items: Albumin, total protein concentration, ALT, AST, ALP, etc.

\textcircled{2} Liver tissue Histology: H&E staining

### Example 4. Confirmation of Enhancement of Treatment Efficacy According to Co-Administration with Colchicine

4-1. Confirmation of Changes in Blood and Serum Levels of Biphenyldimethyl Dicarboxylate (DDB) Alone-Administered Group and Colchicine and Biphenyldimethyl Dicarboxylate (DDB) Co-Administered Group

Among the experimental groups classified by Example 3-3, in a biphenyldimethyl dicarboxylate (DDB) alone-administered group and a colchicine co-administered group by changing the concentration of the biphenyldimethyl dicarboxylate (DDB), changes in levels were confirmed for total five items of albumin levels, ALP, ALT, AST, and total protein concentrations and the results thereof were shown in Table 2 below. At this time, the dose of colchicine administered was all the same at 10 μg/kg.

TABLE 2

| Group | Albumin | ALP | ALT | AST | Total protein |
|---|---|---|---|---|---|
| (n = 7) | (g/dl) | (U/L) | (U/L) | (U/L) | (g/dl) |
| Normal | 2.32 ± 0.10 | 389.09 ± 44.27 | 40.61 ± 7.76 | 69.81 ± 10.30 | 6.08 ± 0.17 |
| Vehicle | 2.30 ± 0.14 | 388.83 ± 52.95 | 34.53 ± 4.22 | 92.07 ± 20.86 | 6.06 ± 0.33 |
| CCl₄ | 2.10 ± 0.14 | 1344.57 ± 318.97 | 1807.63 ± 545.72 | 1740.09 ± 719.02 | 4.92 ± 0.50 |
| DDB | 2.13 ± 0.09 | 1428.90 ± 681.53 | 1543.09 ± 408.03 | 1642.24 ± 764.93 | 5.35 ± 0.28 |
| [25 | | | | | |
| mg/kg] | | | | | |
| Col + | 2.22 ± 0.14 | 1325.80 ± 335.64 | 1199.77 ± 361.07 | 1123.83 ± 412.98 | 5.55 ± 0.30 |
| DDB [25 | | | | | |
| mg/kg] | | | | | |
| Col + | 2.07 ± 0.16 | 1174.00 ± 439.83 | 1113.00 ± 495.19 | 845.23 ± 398.46 | 5.13 ± 0.44 |
| DDB [20 | | | | | |
| mg/kg] | | | | | |
| Col + | 2.25 ± 0.50 | 1250.29 ± 331.90 | 1084.14 ± 268.06 | 815.00 ± 230.09 | 5.17 ± 0.56 |
| DDB | | | | | |
| [10 | | | | | |
| mg/kg] | | | | | |

\textcircled{1} Albumin: Albumin is an item for evaluating the synthesis ability of liver, and as shown in Table 2 and FIG. 1A, it was confirmed that the level which has been reduced by the occurrence of liver damage caused by CCl₄ tended to be recovered in the DDB alone and colchicine co-administered groups.

\textcircled{2} ALP (Alkaline phosphatase): Alkaline phosphatase (ALP) is a liver inflammation level, and as shown in Table 2 and FIG. 2A, it was confirmed that hepatitis occurred by CCl₄ administration, and the level was increased in the biphenyldimethyl dicarboxylate (DDB) alone-administered group, but in the colchicine co-administered group, the inflammation tended to be recovered due to a significant decrease of the level.

\textcircled{3} ALT (GPT): ALT (GPT) is a liver inflammation level, and as shown in Table 2 and FIG. 3A, it was confirmed that the level was significantly increased by CCl₄ administration to cause hepatitis, and in the colchicine co-administered group, the inflammation tended to be recovered due to a significant decrease of the level.

\textcircled{4} AST (GOT): AST (GOT) is a liver inflammation level, and as shown in Table 2 and FIG. 3A, it was confirmed that the level was significantly increased by CCl₄ administration to cause hepatitis, and in the colchicine co-administered group, the inflammation tended to be recovered due to a significant decrease of the level.

\textcircled{5} Total protein: The concentration of the total protein is an item indicating the synthesis ability of the liver, and as shown in Table 2 and FIG. 4A, it was confirmed that the value which has been reduced by CCl₄ administration tended to be recovered in the DDB alone group and the colchicine combination group.

4-2. Confirmation of Changes in Blood and Serum Levels of Silymarin Alone-Administered Group and Silymarin and Colchicine Co-Administered Group

Among the experimental groups classified by Example 3-3, in a silymarin alone-administered group and a colchicine co-administered group, changes in levels were confirmed for total five items of albumin levels, ALP, ALT, AST, and total protein concentrations by changing the concentration of the silymarin, and the results thereof were shown in Table 3 below. At this time, the dose of colchicine administered was all the same at 10 μg/kg.

TABLE 3

| Group | Albumin | ALT | AST | Total protein |
|---|---|---|---|---|
| (n = 7) | (g/dl) | (U/L) | (U/L) | (g/dl) |
| Normal | 2.32 ± 0.10 | 40.61 ± 7.76 | 69.81 ± 10.30 | 6.08 ± 0.17 |
| Vehicle | 2.30 ± 0.14 | 34.53 ± 4.22 | 92.07 ± 20.86 | 6.06 ± 0.33 |
| CCl₄ | 2.10 ± 0.14 | 1807.63 ± 545.72 | 1740.09 ± 719.02 | 4.92 ± 0.50 |
| Silymarin | 2.12 ± 0.21 | 1725.71 ± 406.12 | 1604.57 ± 402.46 | 5.24 ± 0.44 |
| [50 | | | | |
| mg/kg] | | | | |
| Col + | 2.19 ± 0.18 | 1608.36 ± 666.98 | 1560.40 ± 793.89 | 5.53 ± 0.30 |
| Sily [50 | | | | |
| mg/kg] | | | | |
| Col + | 1.95 ± 0.21 | 1377.43 ± 409.90 | 1424.14 ± 741.45 | 4.89 ± 0.46 |
| Sily [30 | | | | |
| mg/kg] | | | | |
| Col + | 2.10 ± 0.14 | 1230.29 ± 389.05 | 1103.57 ± 331.79 | 5.06 ± 0.36 |
| Sily [10 | | | | |
| mg/kg] | | | | |

\textcircled{1} Albumin: As shown in Table 3 and FIG. 1B, it was confirmed that the level which had been reduced due to the occurrence of the liver damage caused by CCl₄ tended to be recovered in the silymarin alone group and the colchicine combination group.

\textcircled{2} ALT (GPT): As shown in Table 3 and FIG. 3B, it was confirmed that the level was significantly increased by CCl₄ administration to cause hepatitis, and in the colchicine co-administered group, the inflammation tended to be recovered due to a significant decrease of the level.

\textcircled{3} AST (GOT): As shown in Table 3 and FIG. 4B, it was confirmed that the level was significantly increased by CCl₄ administration to cause hepatitis, and in the colchicine co-administered group, the inflammation tended to be recovered due to a significant decrease of the level.

\textcircled{4} Total protein: As shown in Table 3 and FIG. 5B, it was confirmed that the level which had been reduced by CCl₄ administration tended to be recovered in the silymarin alone group and the colchicine combination group.

4-3. Confirmation of Changes in Blood and Serum Levels in Colchicine and UDCA Co-Administered Group

Among the experimental groups classified by Example 3-3, in a colchicine and UDCA co-administered group, changes in levels were confirmed for total five items of albumin levels, ALP, ALT, AST, and total protein concentrations and the results thereof were shown in Table 4 below. At this time, the dose of colchicine administered was all the same at 10 μg/kg.

TABLE 4

| Group | Albumin | ALP | ALT | AST | Total protein |
|---|---|---|---|---|---|
| (n = 7) | (g/dl) | (U/L) | (U/L) | (U/L) | (g/dl) |
| Normal | 2.32 ± 0.10 | 389.09 ± 44.27 | 40.61 ± 7.76 | 69.81 ± 10.30 | 6.08 ± 0.17 |
| Vehicle | 2.30 ± 0.14 | 388.83 ± 52.95 | 34.53 ± 4.22 | 92.07 ± 20.86 | 6.06 ± 0.33 |
| CCl₄ | 2.10 ± 0.14 | 1344.57 ± 318.97 | 1807.63 ± 545.72 | 1740.09 ± 719.02 | 4.92 ± 0.50 |
| Col + | 2.27 ± 0.38 | 1129.00 ± 718.34 | 905.14 ± 427.26 | 837.00 ± 552.87 | 5.51 ± 0.42 |
| UDCA | | | | | |
| [30 | | | | | |
| mg/kg] | | | | | |

\textcircled{1} Albumin: As shown in Table 4 and FIG. 1C, it was confirmed that the level which had been reduced due to the occurrence of the liver damage caused by CCl₄ tended to be recovered in the colchicine combination group.

\textcircled{2} ALP (Alkaline phosphatase): As shown in Table 4 and FIG. 2C, it was confirmed that hepatitis occurred by CCl₄ administration and a significant reduction effect was shown in the colchicine combination group.

\textcircled{3} ALT (GPT): As shown in Table 4 and FIG. 3C, it was confirmed that the level was significantly increased by CCl₄ administration to cause hepatitis, and in the colchicine co-administered group, the inflammation tended to be recovered due to a significant decrease of the level.

\textcircled{4} AST (GOT): As shown in Table 4 and FIG. 4C, it was confirmed that the level was significantly increased by CCl₄ administration to cause hepatitis, and in the colchicine co-administered group, the inflammation tended to be recovered due to a significant decrease of the level.

\textcircled{5} Total protein: As shown in Table 4 and FIG. 5C, it was confirmed that the level which had been reduced by CCl₄ administration tended to be recovered in the colchicine combination group.

4-4. Hydroxyproline Assay Quantitation

Hydroxyproline assay was performed for each group to confirm the level of fibrin accumulation in the liver.

TABLE 5

| | Hydroxyproline |
|---|---|
| Group | [μg/g of liver] |
| Normal | 274.81 ± 17.49 |
| Vehicle | 247.86 ± 23.10 |
| CCl₄ [0.4 g/kg] | 336.36 ± 18.82 |
| DDB [25 mg/kg \| | 317.74 ± 9.24 |
| Colchicine [10 μg/kg] + DDB [25 mg/kg] | 314.72 ± 21.66 |
| Colchicine [10 μg/kg] + DDB [20 mg/kg] | 313.94 ± 23.14 |
| Colchicine [10 μg/kg] + DDB [10 mg/kg] | 302.52 ± 10.20 |

As shown in Table 5 and FIG. 6A, a significant reduction effect in fibrin was shown in the colchicine and DDB co-administered group as compared with the DDB alone administered group.

TABLE 6

| | Hydroxyproline |
|---|---|
| Group | [μg/g of liver] |
| Normal | 274.81 ± 17.49 |
| Vehicle | 247.86 ± 23.10 |
| CCl₄ [0.4 g/kg] | 336.36 ± 18.82 |
| Silymarin [50 mg/kg] | 310.48 ± 15.83 |
| Colchicine [10 μg/kg] + Silymarin [50 mg/kg] | 309.91 ± 11.09 |
| Colchicine [10 μg/kg] + Silymarin [30 mg/kg] | 308.66 ± 16.54 |
| Colchicine [10 μg/kg] + Silymarin [10 mg/kg] | 302.85 ± 16.28 |

As shown in Table 6 and FIG. 6A, a significant reduction effect in fibrin was shown in the colchicine and silymarin co-administered group as compared with the silymarin alone administered group.

TABLE 7

| | Hydroxyproline |
|---|---|
| Group | [μg/g of liver] |
| Normal | 274.81 ± 17.49 |
| Vehicle | 247.86 ± 23.10 |
| CCl₄ [0.4 g/kg] | 336.36 ± 18.82 |
| Colchicine [10 μg/kg] + UDCA [30 mg/kg] | 302.02 ± 12.55 |

As shown in Table 7 and FIG. 6C, a significant reduction effect in fibrin was shown in the colchicine and silymarin co-administered group as compared with the silymarin alone administered group.

4-5. Liver Histopathological Examination (H&E Staining)

In each group, when the pathological characteristics for fatty necrosis, ballooning, fatty change, and fibrosis may be identified according to severity of disease, it was evaluated by assigning 0 point for no disease (NAD), 1 point for mild symptoms, 2 points for moderate symptoms, and 3 points for severe symptoms.

TABLE 8

| | Fatty | Ballooning | Fatty | | Total |
|---|---|---|---|---|---|
| | necrosis | degeneration | change | Fibrosis | Score |
| Normal | 0 | 0 | 0 | 0 | 0 |
| Vesicle | 1 | 0 | 0 | 0 | 1 |
| CCl₄ | 1 | 3 | 1 | 3 | 8 |
| DDB [25 mg] | 1 | 3 | 1 | 2 | 7 |
| Col + DDB [25 mg] | 1 | 1 | 1 | 2 | 5 |
| Col + DDB [20 mg] | 1 | 2 | 1 | 2 | 6 |
| Col + DDB [10 mg] | 1 | 1 | 1 | 2 | 5 |
| Silymarin [50 mg] | 1 | 3 | 1 | 2 | 7 |
| Col + Silymarin [50 | 2 | 2 | 1 | 2 | 7 |
| mg] | | | | | |
| Col + Silymarin [30 | 1 | 2 | 0 | 3 | 6 |
| mg] | | | | | |
| Col + Silymarin [10 | 0 | 2 | 1 | 3 | 6 |
| mg] | | | | | |
| Col + UDCA [30 mg] | 0 | 1 | 1 | 2 | 4 |

As a result, as shown in Table 8 and FIG. 7, it was confirmed that colchicine and biphenyldimethyl dicarboxylate (DDB), silymarin, or UDCA were co-administered to further improve pathologic findings.

The aforementioned description of the present invention is to be exemplified, and it will be understood by those skilled in the art that the technical spirit or required features of the present invention can be easily modified in other detailed forms without changing. Therefore, it should be appreciated that the aforementioned embodiments are illustrative in all aspects and are not restricted.

1. A pharmaceutical composition for treatment of liver disease comprising colchicine and biphenyldimethyl dicarboxylate (DDB) as active ingredients.

2. The pharmaceutical composition for treatment of liver disease of claim 1, wherein contents of the ingredients included in the pharmaceutical composition are 10 to 120 μg/kg of the colchicine and 5 to 30 mg/kg of the biphenyldimethyl dicarboxylate.

3. The pharmaceutical composition for treatment of liver disease of claim 1, wherein the liver disease is at least one selected from liver fibrosis, liver cirrhosis, liver cancer, and inflammatory liver disease.

4. The pharmaceutical composition for treatment of liver disease of claim 3, wherein the inflammatory liver disease is at least one selected from hepatitis, acute hepatitis, chronic hepatitis, alcoholic hepatitis, non-alcoholic hepatitis, subacute hepatitis, viral hepatitis, toxic liver disease, liver abscess, granulomatous hepatitis, autoimmune hepatitis, and lupus hepatitis.

5. The pharmaceutical composition for treatment of liver disease of claim 1, wherein the pharmaceutical composition is in any one formulation selected from the group consisting of powders, granules, tablets, capsules, suspensions, emulsions, syrups, aerosols, external preparations, suppositories, and injection solutions.

6. The pharmaceutical composition for treatment of liver disease of claim 1, wherein the pharmaceutical composition further comprises at least one ingredient selected from the group consisting of lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc and magnesium stearate.

7. The pharmaceutical composition for treatment of liver disease of claim 1, wherein the pharmaceutical composition further comprises at least one ingredient selected from the group consisting of thiamine, riboflavin, niacin, pantothenic acid, pyridoxine, cobalamin, vitamin C, vitamin D, and vitamin E.

8. A pharmaceutical composition for enhancing the therapy of liver disease comprising colchicine and biphenyldimethyl dicarboxylate (DDB) as active ingredients.

9. The pharmaceutical composition for enhancing the therapy of liver disease claim 8, wherein contents of the ingredients included in the pharmaceutical composition are 10 to 120 μg/kg of the colchicine and 5 to 30 mg/kg of the biphenyldimethyl dicarboxylate.

10. The pharmaceutical composition for enhancing the therapy of liver disease of claim 8, wherein the liver disease is at least one selected from liver fibrosis, liver cirrhosis, liver cancer, and inflammatory liver disease.

11. The pharmaceutical composition for enhancing the therapy of liver disease of claim 10, wherein the inflammatory liver disease is at least one selected from hepatitis, acute hepatitis, chronic hepatitis, alcoholic hepatitis, non-alcoholic hepatitis, subacute hepatitis, viral hepatitis, toxic liver disease, liver abscess, granulomatous hepatitis, autoimmune hepatitis, and lupus hepatitis.

12. The pharmaceutical composition for enhancing the therapy of liver disease of claim 8, wherein the pharmaceutical composition is in any one formulation selected from the group consisting of powders, granules, tablets, capsules, suspensions, emulsions, syrups, aerosols, external preparations, suppositories, and injection solutions.

13. The pharmaceutical composition for enhancing the therapy of liver disease of claim 8, wherein the pharmaceutical composition further comprises at least one ingredient selected from the group consisting of lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc and magnesium stearate.

14. The pharmaceutical composition for enhancing the therapy of liver disease of claim 8, wherein the pharmaceutical composition further comprises at least one ingredient selected from the group consisting of thiamine, riboflavin, niacin, pantothenic acid, pyridoxine, cobalamin, vitamin C, vitamin D, and vitamin E.
